(19) United States
(12) Patent Application Publication
(10) Pub. No.: US 2017/0220710 A1
(43) Pub. Date: Aug. 3, 2017

(54) QUANTITATIVE METHODS FOR HETEROGENEOUS SAMPLE COMPOSITION DETERMINATION AND BIOCHEMICAL CHARACTERIZATION

(71) Applicants: John David Chung, Kelseyville, CA (US); Peter Lee Zhan, Foster City, CA (US)

(72) Inventors: John David Chung, Kelseyville, CA (US); Peter Lee Zhan, Foster City, CA (US)

(21) Appl. No.: 15/014,968

(22) Filed: Feb. 3, 2016

(51) Int. Cl. G06F 17/50; G06F 19/16; G01N 33/68; G06F 19/12

(52) U.S. Cl. CPC G06F 17/5009; G06F 19/12; G06F 19/16; G01N 33/6854

(57) ABSTRACT

Methods involving the use of mathematical models of competitive ligand-receptor binding to characterize mixtures of ligands in terms of compositions and properties of the component ligands have been developed. The associated mathematical equations explicitly relate component ligand physical-chemical properties and mole fractions to measurable properties of the mixture including steady state binding activity, 1/K_{d,apparent} or equivalently 1/EC50, and kinetic rate constants k_{on,apparent} and k_{off,apparent} allowing; 1) component ligand physical property determination and 2) mixture property predictions. Additionally, mathematical equations accounting for combinatorial considerations associated with ligand assembly are used to compute ligand mole fractions. The utility of the methods developed is demonstrated using published experimental ligand-receptor binding data obtained from mixtures of afucosylated antibodies that bind FcγRIIIa (CD16a) to: 1) extract component ligand physical property information that has hitherto evaded researchers 2) predict experimental observations and 3) provide explanations for unresolved experimental observations.

### CROSS REFERENCE

This application claims the benefit of PPA Ser. No. 62/176,362, filed Feb. 16, 2015 by the present inventors, which is incorporated by reference.

### BACKGROUND

### Prior Art

### Nonpatent Literature Documents

Chung S., Quarmby V., Gao, X. et al., “Quantitative evaluation of fucose reducing effects in humanized antibody on Fcγ receptor binding and antibody-dependent cell-mediated cytotoxicity activities” mAbs 4:3, 326-340, 2012.

Biologics produced by mammalian cell culture are inherently heterogeneous due to non-uniform glycosylation. Since glycosylation is known to influence drug efficacy, characterizing glycoform heterogeneity is important for drug quality and safety. However due to the large size of glycoproteins, characterizing mixtures of glycoproteins in terms of the identities, the quantities and the specific activities of the biologically active glycoforms continues to challenge researchers. Direct experimental methods for reliably identifying and quantitating ligands in mixtures are challenged by sensitivity issues associated with reliably detecting and quantitating relatively small differences that might exist between molecules. Structure-activity relationships between glycoform composition and biochemical activity are difficult to identify amidst mixtures of glycoproteins.

FIGURE Prior-Art highlights the general approach used to characterize therapeutic glycoprotein ligands such as antibodies in terms of their biochemical activity and carbohydrate structures. Determining the biochemical activity of a mixture of ligands is generally straightforward using standard ligand-receptor binding assays. In contrast, current methods of glycoform analysis requires that the carbohydrate moieties attached to the ligands in the mixture be physically removed from the amino acid backbone of the glycoprotein, typically by enzymatic digestion, followed by the analysis of the released carbohydrate. Since the carbohydrates that define the different glycoforms are physically detached from the proteins, glycoform analysis provides sample average information on a heterogeneous population of glycoforms. However due to the complexity of most therapeutic glycoproteins, this information can be used to deduce the molar concentrations of the different glycoforms for only the simplest of ligands. Accordingly many therapeutic glycoprotein ligands are characterized in terms of the concentrations and the composition of attached carbohydrates rather than that of the glycoforms or glycoproteins.

The limitations of current approaches for glycoprotein characterization are illustrated with therapeutic monoclonal antibodies that rely on Fc mediated effector function for biological activity. Anti-cancer IgG1 therapeutics have been shown to possess differential Fc mediated biochemical activity as the result of their afucosylated Fc glycan content. The presence of a core fucose molecule on the carbohydrate attached to the conserved Asn²⁹⁷ residue of IgG1 has been shown to dramatically reduce the affinity of the Ig Fc region for the FcγRIIIa (CD16a) receptor and in vitro antibody-dependent cellular cytotoxicity (ADCC) activity. Standard glycoform analysis provides the sample average fraction of Fc glycans that are afucosylated or devoid of a core fucose molecules, denoted by p, but it cannot provide information on the molar concentrations of the different afucosylated antibody glycoforms. An antibody molecule consists of two heavy chains each with a potential afucosylation site so that three different afucosylated antibody glycoforms exist with each form differentiated by the number of afucosylated Fc glycans; the homogeneous afucosylated antibody form containing two afucosylated Fc heavy chains, the hemi-afucosylated antibody form containing one afucosylated Fc heavy chain and the homogeneous fucosylated antibody form containing zero afucosylated Fc heavy chains.

The existence of three different afucosylated antibody glycoforms makes it impossible to uniquely characterize a mixture of afucosylated antibody ligands by the afucosylated Fc glycan fraction metric p. Mixtures of antibodies with the same mixture average afucosylated Fc glycan content can have very different glycoform molar concentrations. Likewise, samples that exhibit similar in vitro biochemical activities can have very different molar concentrations of the different afucosylated antibody glycoforms. Since the antibody is the physical entity that mediates in vivo efficacy, knowledge of the molar concentrations of the different afucosylated antibodies is fundamental to proper product characterization. Drug quality has the potential to suffer from this lack of information since different manufacturers of the “same” glycoprotein must argue for product similarity without basic information on the molar concentrations and the specific activities of the biochemically active glycoforms in their product.

The inability to obtain molar concentration and specific activity information of the important biologically active ligands in a mixture has motivated empirical efforts to correlate structure and activity. These efforts are not without difficulty. The bottom box in FIGURE Prior-Art highlights an empirical analysis of antibody afucosylation by Chung and coworkers (Chung et al. 2012). Chung and coworkers applied linear regression analysis to empirically correlate ELISA-derived FcγRIIIa receptor binding activity, defined as 1/EC50 where EC50 denotes the experimental ligand concentration that induces a ½ maximal experimental output response in a ligand-receptor binding assay, with mixture afucosylation content or the fraction of Fc glycans that are afucosylated p. However a unique correlation could not be established. Instead, many different empirical correlations were found so that samples with similar afucosylation content could have very different activity and samples with similar activity could have very different afucosylation content. Empirical analysis could not provide explanations for these differences.

The study of Chung and coworkers highlights a danger associated with the overreliance on sample average metrics of protein quality such asp in lieu of the molar concentrations of the underlying glycoproteins. The attempt to linearly correlate activity with Fc afucosylation content p necessarily assumes that p adequately characterizes a mixture of afucosylated antibodies. However, uniquely specifying the composition of a three component mixture requires that two of the three mole fractions are specified. Therefore p cannot in general substitute for the two mole fractions required to adequately specify a ternary afucosylated antibody mixture. The sole use of p to characterize antibody fucosylation content is not expected to be adequate.

### SUMMARY

Methods for characterizing mixtures of glycoproteins in terms of the compositions and the biochemical properties of constituent glycoproteins using mathematical models of competitive ligand-receptor binding have been developed. The mathematical equations or structure imposed by the competitive ligand receptor mechanism on a mixture of glycoprotein ligands allows constituent glycoproteins information to be deduced from mixture property information providing a means to characterize constituent glycoproteins that cannot be isolated in pure form. The methods also provide the means to predict mixture properties when provided with constituent ligand property information. The methods are applied to characterize mixtures of monoclonal antibodies comprised of antibodies with different afucosylated Fc glycan content allowing the dissociation equilibrium constant of the hemi-afucosylated IgG1 form to be deduced from published Ig Fc-FcγRIIIa (CD16a) ligand-receptor binding data gathered by ELISA. To date, this parameter has evaded researchers. Although the methods are used to analyze antibody mixtures in terms of their Fc mediated binding activity, the methods developed are not limited to this specific assay format or a specific choice of ligand-receptor pair, but are applicable to any assay format capable of measuring ligand-receptor or binary protein-protein binding that is governed by competitive binding.

### Advantages

From the description above, a number of advantages of the methods developed over Prior-Art become evident:
a. the means to determine the properties of the constituent ligands in a mixture of ligands, including the equilibrium constants and the forward and reverse kinetic rate constants of the associated ligand-receptor binding reaction,
b. the means to predict the properties of mixtures of ligands including the apparent equilibrium, K_{d,apparent}, and the apparent rate constants, k_{on,apparent} and k_{off,apparent}, using constituent ligand properties and compositions,
c. the means to predict the steady state receptor binding curves for mixtures of ligands from constituent ligand properties and compositions,
d. the means to determine the molar compositions of constituent ligands in mixtures of ligands.

### DRAWINGS—FIGURES

FIG. 1: Flow chart for computing ligand properties.

FIG. 2: Computing the dissociation equilibrium constant K_{AF}.

FIG. 3: Computing the dissociation equilibrium constant K_{A}.

FIG. 4: Computing kinetic rate constants k_{on,AF} and k_{off,AF}.

FIG. 5: Flow chart for computing mixture properties.

FIG. 6: Computing 1/K_{d,apparent}.

FIG. 7: Computing receptor binding dose-response curves.

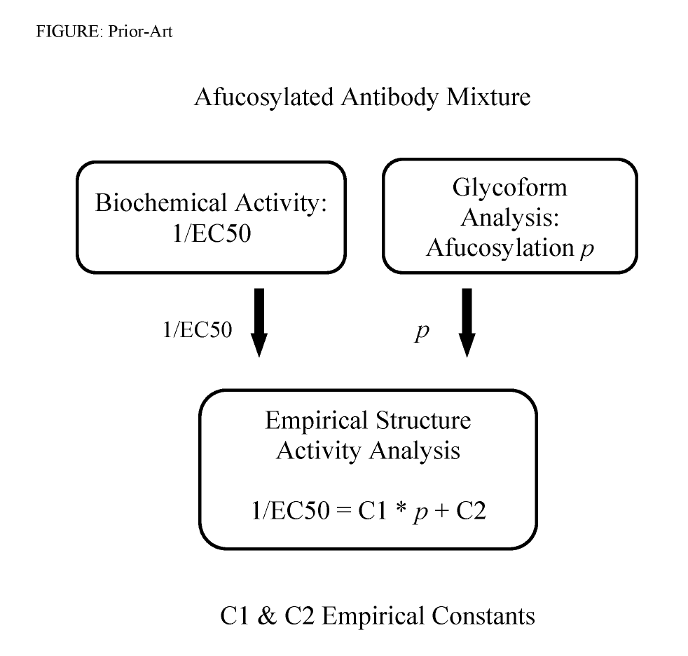

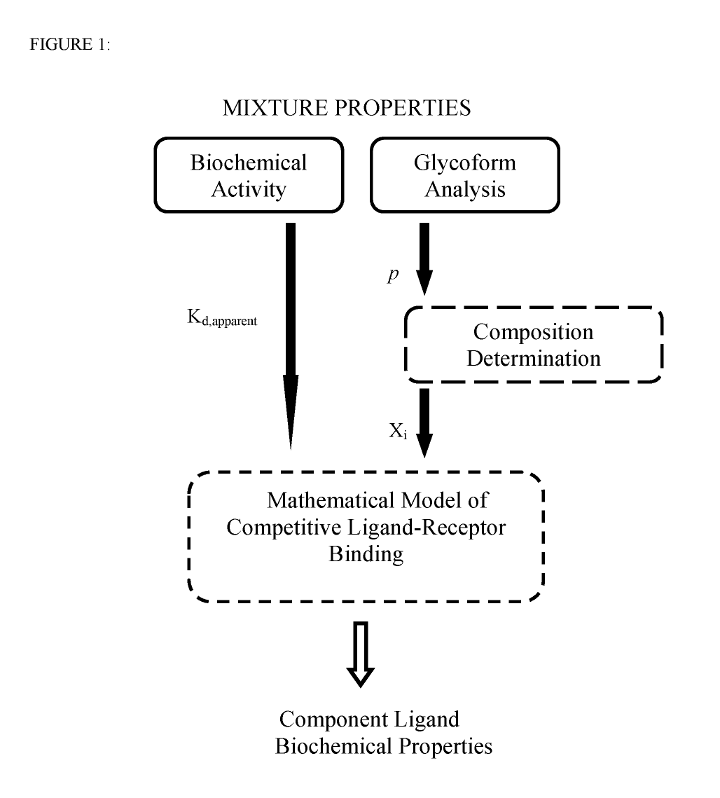

### DETAILED DESCRIPTION—FIRST EMBODIMENT—FIG. 1

FIG. 1 shows the general flowchart of how mechanistic mathematical models of competitive ligand-receptor binding are used to characterize mixtures of glycoprotein ligands showing steps that are common with current methods, with solid outlines, and the steps that involved the use of mechanism-based mathematical models, with dashes outlines. The top two “boxes” represent two common orthogonal or independent methods used to characterize mixtures of glycoprotein ligands; biochemical activity and glycoform or carbohydrate analysis. The arrows joining the boxes denote the flow of information or data.

Measures of biochemical activity may be steady state or kinetic in nature. Steady state measures of biochemical activity include 1/EC50, or equivalently 1/K_{d,apparent}, obtained from steady state ligand-receptor binding curves where EC50 and K_{d,apparent} denote the experimental ligand concentration that induces a ½ maximal experimental output in a ligand-receptor binding assay. As discussed later, kinetic rate constants such as k_{on,apparent} also measure biochemical activity. The subscript “apparent” denotes the fact that in general the value of K_{d} and kₒₙ obtained from mixtures will depend on mixture composition. As such K_{d} and kₒₙ for mixtures are denoted K_{d,apparent} and k_{on,apparent} respectively. With pure samples, composition is no longer variable and the subscript “apparent” is omitted.

In addition to biochemical activity, carbohydrate composition information is routinely available for glycoprotein ligands of industrial importance. Glycans or carbohydrates must be physically removed from the amino acid backbone of the glycoprotein before their compositions can be determined so that carbohydrate composition data generally provides sample average information on glycoform structure and composition. For mixtures comprising the three afucosylated antibody glycoforms, glycoform or carbohydrate analysis provides the overall fraction of Fc glycans that are afucosylated, denoted p.

The dashed boxes in FIG. 1 show how mathematical models are used to extract additional biochemical property information from existing activity and glycoform analysis data. The bottom box depicts the use of mathematical models of competitive ligand-receptor modeling to compute component ligand properties such as the dissociation equilibrium constant Kᵢ from mixture biochemical activity and mole fractions Xᵢ's. K_{d,apparent} is obtained directly from experimental measurement of receptor binding activity. Mole fractions or mixture composition are obtained by many methods depending on the specific ligand-receptor system including; direct experimental measurements, mass balances or statistical distributions or combinations of such methods.

The term “competitive” used in the phrase “mathematical model of competitive ligand-receptor binding” refers specifically to the scientifically accepted use of the term “competitive” in the phrases “competition binding” and “competitive inhibition” in the fields of Biochemistry and Biophysics. Competitive binding is mutually exclusive in nature requiring that the binding of ligand i to a specific receptor site is sufficient to prevent the binding of a different ligand j to the same receptor binding site and vice-versa. Thus, the different ligands compete for binding to common receptor binding sites. These concepts are described more formally using mathematics in the following sections.

### Steady State Data Analysis

For a system of m ligands L₁, L₂, . . . , Lₘ₋₁, Lₘ, that compete for binding to a common receptor R, the following m chemical equations apply:

L₁+R [symbol] RL₁, L₂+R [symbol] RL₂, Lₘ+R [symbol] RLₘ.

The general mathematical equation imposed on this system at steady state by the competitive binding mechanism is given by equation (1):

$$activity = \frac{1}{EC 50} = \frac{1}{{K}_{d , apparent}} = \frac{{X}_{1}}{{K}_{1}} + \frac{{X}_{2}}{{K}_{2}} + … + \frac{{X}_{m}}{{K}_{m}} \qquad ( 1 )$$

with mole fractions Xᵢ:

$${X}_{i} = \frac{[ {L}_{i} ]}{{\sum }_{j = 1}^{m} [ {L}_{j} ]} .$$

[Lᵢ] and Xᵢ denote the molar concentration and the mole fraction of unbound component ligand i. Equation 1 is obtained by combining and algebraically manipulating; the definition of the dissociation equilibrium constant for ligand i, Kᵢ, the definition of the apparent dissociation equilibrium constant for mixtures, K_{d,apparent}, and the molar balances on ligand and ligand-receptor complex:

$${K}_{{L}_{1}} = \frac{[ R ] [ {L}_{1} ]}{[ {RL}_{1} ]} , {K}_{{L}_{2}} = \frac{[ R ] [ {L}_{2} ]}{[ {RL}_{2} ]} , … , {K}_{{L}_{n}} = \frac{[ R ] [ {L}_{m} ]}{[ {RL}_{m} ]} and$$

$${K}_{d , apparent} = \frac{{[ R ] [ L ]}_{total}}{{[ RL ]}_{total}} ,$$

with molar balances:

[L]ₜₒₜₐₗ=[L₁]+[L₂]+ . . . +[Lₘ] (ligand balance)

[RL]ₜₒₜₐₗ=[RL₁]+[RL₂]+ . . . +[RLₘ] (ligand-receptor complex)

[R] denotes the unbound molar concentration of receptor R. [RLᵢ] denotes the molar concentration of ligand i-receptor complex.

Equation (1) relates experimental receptor binding activity, defined as 1/EC50, to component ligand mole fractions, Xᵢ's, and dissociation equilibrium constants, s. In terms of the competitive binding model, 1/EC50 is identically the model parameter 1/K_{d,apparent}. Equation (1) reveals that mixture activity, 1/K_{d,apparent}, is the sum of the specific activities of the component ligands, 1/Kᵢ's, weighted by their respective mole fractions. Accordingly, equation (1) provides the means to compute the component ligand dissociation equilibrium constants Kᵢ's, or the corresponding binding constants given by 1/Kᵢ's when provided with the appropriate composition and mixture activity data.

Equation (2) is the specific form of equation (1) that describes the antibody afucosylation system comprising three different antibody glycoform ligands:

$$activity = \frac{1}{EC 50} = \frac{1}{{K}_{d , apparent}} = \frac{{X}_{A}}{{K}_{A}} + \frac{{X}_{F}}{{K}_{F}} + \frac{{X}_{AF}}{{K}_{AF}} . \qquad ( 2 )$$

Equation (2) may be obtained directly from equation (1) using the appropriate subscripts. In terms of this ternary antibody system, K_{d,apparent} is defined by:

$${K}_{d , apparent} = \frac{{[ R ] [ Ab ]}_{total}}{{[ RAb ]}_{total}}$$

with [Ab]ₜₒₜₐₗ and [RAb]ₜₒₜₐₗ denoting the molar concentration of total unbound antibody and the sum of the molar concentrations of all three antibody-receptor complexes respectively. The three afucosylated antibody glycoforms are differentiated by their afucosylated Fc glycan content with the homogeneous fucosylated antibody F containing zero afucosylated Fc glycans, the hemi-afucosylated antibody AF containing one afucosylated Fc glycan, and the homogeneous afucosylated antibody A containing two afucosylated Fc glycans. The Fc region of the three afucosylated antibody ligands compete for binding to the FcγRIIIa (CD16a) receptor with the associated dissociation equilibrium constants K_{A}, K_{AF} and K_{F}, where the subscripts denote the specific glycoforms. X_{A}, X_{F} and X_{AF} denote the three antibody mole fractions with the subscripts denoting the specific glycoform.

Use of the mathematical models described to analyze experimental receptor binding data requires that the unbound concentrations of antibodies and receptor are known. When such data are not readily available, the general molar excess of ligand over receptor allows the ligand concentrations appearing in the equations to be approximated by the molar ligand concentration added to the experimental samples. Unless otherwise noted, the numerical values used for ligand or antibody concentrations are assumed to be equal to the ligand or antibody concentrations added to the sample.

### Kinetic Data Analysis

The general mathematical constraint imposed by the competitive ligand-receptor binding mechanism on the forward kinetic rate constants of a ligand mixture comprises m components is given by:

kₒₙ=k_{on,apparent}=X₁k_{on,1}+X₂k_{on,2}+ . . . +Xₘ₋₁k_{on,m-1}+Xₘk_{on,m} (3).

Equation (3) reveals that the forward rate constant for the mixture, k_{on,apparent}, is the sum of the forward rate constants of the component ligands, k_{on,i}s, weighted by their respective mole fractions. For the afucosylated antibody system involving three glycoform ligands, equation (3) simplifies to:

kₒₙ=k_{on,apparent}=X_{A}k_{on,A}+X_{F}k_{on,F}+X_{AF}k_{on,AF} (4)

with the component ligand subscripts altered accordingly.

Equation (3) is obtained by noting that the overall rate of ligand-receptor binding is the sum of the rates of ligand-receptor binding of the component ligands:

$${r}_{on} = {{k}_{on , apparent} [ L ]}_{total} [ R ] = {\sum }_{i = 1}^{m} {r}_{on , i} .$$

Combining the above equation with component ligand mass action rate laws,

r_{on,i}=k_{on,i}[Lᵢ][R] i=1,2, . . . ,m

and solving for k_{on,apparent} yields equation (3). Equation (4) follows immediately from equation (3) with m=3.

The reverse kinetic rate constants, k_{off,i}, for the component ligands can be computed using the dissociation equilibrium constant Kᵢ and the forward rate constant k_{on,i} using the well-known equation:

$${K}_{i} = \frac{{k}_{off , i}}{{k}_{on , i}} . \qquad ( 5 )$$

Alternatively, performing an analysis similar to that used to derive equation (3), one can arrive at:

$${k}_{off} = {k}_{off , apparent} = {f}_{1}^{*} {k}_{off , 1} + {f}_{2}^{*} {k}_{off , 2} + {f}_{m - 1}^{*} {k}_{off , m - 1} + {f}_{m}^{*} {k}_{off , m} \\ {f}_{i}^{*} = \frac{[ {RL}_{i} ]}{{[ RL ]}_{total}} . \qquad ( 6 )$$

fᵢ* denotes the fraction of all the bound receptors [RL]ₜₒₜₐₗ that include ligand i in complex with receptor R. The ternary component analog of equation (6) applicable to the antibody afucosylation system is given by:

k_{off}=k_{off,apparent}=f_{A}*k_{off,A}+f_{F}*k_{off,F}+f*_{AF}k_{off,AF} (7).

### Statistical and Combinatorial Considerations

The primary sequence of many glycoform ligands can often be complex with ligands comprising multiple subunits. Therefore in general, glycoform molar compositions cannot be deduced from carbohydrate or glycan composition data. However when a ligand comprises more than one subunit with the same primary sequence, with the primary sequence containing a glycosylation site, combinatorial considerations can be used to compute the mole fractions of the glycoform variants of the glycoprotein.

The compositions of the afucosylated antibody glycoforms may be computed using the binomial distribution with n=2. Antibody assembly involves the dimerization of two antibody heavy chains with identical amino acid sequence. Each antibody heavy chain possesses an Fc bound glycan with one potential fucosylation site. Fucosylated antibody heavy chains have a core fucose molecule attached to the base of the glycan bound to the conserved Asn²⁹⁷ glycosylation site. Afucosylated antibody heavy chains are devoid of said fucose molecule. The probability that a heavy chain will be afucosylated is given by the fraction of Fc glycan that are afucosylated or p. Therefore the binomial distribution with n=2 can be used to compute the mole fractions of the three afucosylated antibody glycoforms, X_{F}, X_{A} and X_{AF}, in accordance with equation (8):

X_{A}=p²

X_{F}=(1−p)²

X_{AF}=2p(1−p) (8).

Combining equation (8) with equation (2) yields:

$$activity = \frac{1}{EC 50} = \frac{1}{{K}_{d , apparent}} = \frac{{p}^{2}}{{K}_{A}} + \frac{2 ( p - {p}^{2} )}{{K}_{AF}} + \frac{{( 1 - p )}^{2}}{{K}_{F}} . \qquad ( 9 )$$

Similarly, combining equation (8) and equation (4) yields:

kₒₙ=k_{on,apparent}=p²k_{on,A}+(1−p)²k_{on,F}+2(p−p²)k_{on,AF} (10)

Equation (8) reveals that pure samples of homogeneous fucosylated and afucosylated antibodies are obtained in the limiting cases when the fraction of Fc glycans that are afucosylated p approaches either zero or unity respectively. When the afucosylated Fc glycan fraction p→0, the mixture for all practical purposes is considered to be a “pure” homogeneous fucosylated antibody sample and X_{F}→1. Similarly when p→1, the mixture for all practical purposed is considered to be a “pure” homogeneous afucosylated antibody sample and X_{A}→1.

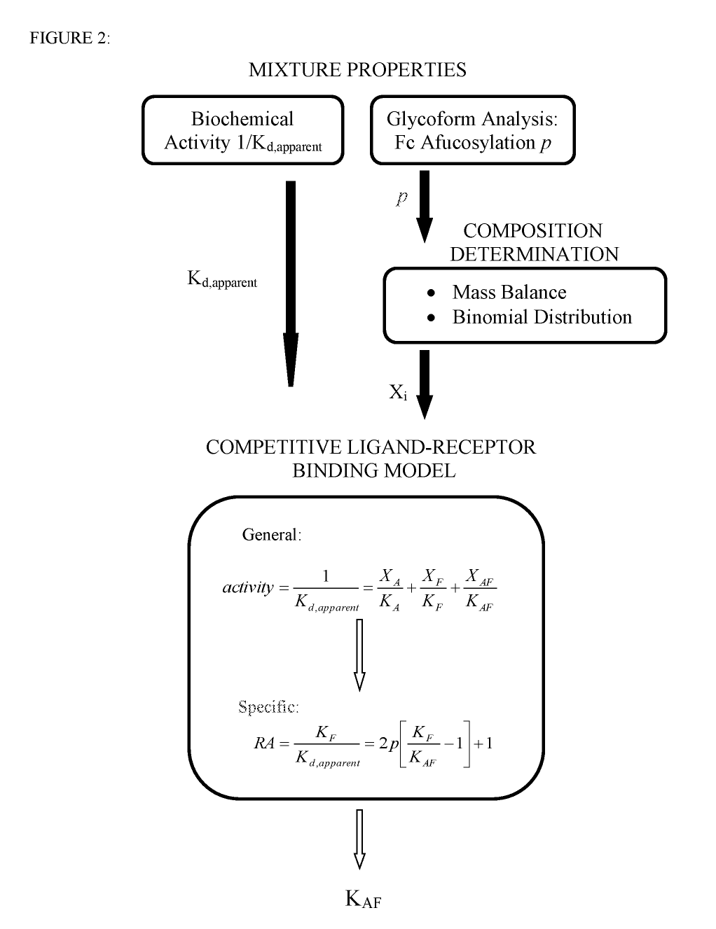

### OPERATION—FIRST EMBODIMENT—FIGS. 2-4

The computational flowchart in FIG. 1 shows the general flow of information involved in using mathematical models to computing component ligand properties from experimental data obtained from mixtures. The general approach is summarized in the following steps:
1) acquire mixture property information such as K_{d,apparent}, by direct experimental measurements,
2) acquire mixture compositions or mole fractions using: direct measurement, statistical distributions, mass balances or combinations of these elements,
3) obtain the specific form the general mathematical model of competitive ligand-receptor binding based on experimental considerations
4) use mathematical model to compute component ligand property.
   These steps allow component ligand properties to be computed from data obtained from mixtures of ligands. When a component ligand cannot be isolated in pure form, the steps outlined provide a means to determine its' properties. Such a means does not currently exist. When a component ligand can be isolate in pure form, determining its' properties is straightforward. The methods comprising the steps listed assume that the properties of glycoforms that are available in pure form are available. The term “pure” refers to a sample of an antibody typically in excess of 95% on a molar basis since 100% purity is rarely achieved in practice.

### Example 1: Computing K_{AF}

FIG. 2 shows the flowchart used to compute K_{AF} for the hemi-afucosylated antibody. Afucosylated Fc glycan content measured by p are used to compute glycoform mole fractions using equation (8). Composition and mixture activities are then used with mathematical models of competitive ligand-receptor binding to compute component ligand properties. The appropriate mathematical models used to compute K_{AF} from the data published by Chung and coworkers (2012) for low p mixtures are given by:

$$\begin{matrix}activity = \frac{1}{EC 50} = \frac{1}{{K}_{d , apparent}} = 2 p [ \frac{1}{{K}_{AF}} - \frac{1}{{K}_{F}} ] + \frac{1}{{K}_{F}} \\ ( p \leq 0.1 ) & ( 11 ) \\ RA = \frac{EC {50}_{F}}{EC 50} = \frac{{K}_{F}}{{K}_{d , apparent}} = 2 p [ \frac{{K}_{F}}{{K}_{AF}} - 1 ] + 1 \\ ( p \leq 0.1 ) . & ( 12 )\end{matrix}$$

Equation (12) is obtain from equation (11) by dividing equation (11) by 1/K_{F}. Equation (11) is obtained directly from equation (9) by noting that when p≦0.1, p² terms may be neglected and equation (9) simplifies to yield equation (11).

Equations (11) predicts that activity or 1/K_{d,apparent} will scale linearly with p when p≦0.1 and that the slope and the y-intercept associated with this linear relationship are given by 2[1/K_{AF}−1/K_{F}] and 1/K_{F} respectively. Similarly, the slope and the y-intercept associated with equation (12) are given by the terms 2[K_{F}/K_{AF}−1] and 1 respectively. Therefore the existence of a linear relationship between mixture activity and p when p≦0.1 can be used to compute K_{AF}.

Chung and coworkers (2012) reported on the existence of an empirical linear correlation between relative activity (RA), obtained from ELISA's measuring antibody Fc-FcγRIIIa F158 binding, and afucosylated Fc glycan fraction p for IgG1 mixtures characterized by p≦0.1. Experimental RA data is obtained by dividing mixture activity, 1/K_{d,apparent}, by the activity of pure homogeneous fucosylated antibodies or 1/K_{F}. Experimental RA data as defined is given by K_{d,apparent}/K_{F} in terms of the model parameters. Note that equations (11) and (12) theoretically predict the existence of a linear relationship between activity and afucosylation content. Therefore equation (12) is the appropriate mathematical model for analyzing the experimental relative activity data of Chung and coworkers.

Using the slope value of the empirical linear correlation reported by Chung and coworkers, 80, K_{AF} can be computed:

$$slope = 80 = 2 [ \frac{{K}_{F}}{{K}_{AF}} - 1 ] = 2 [ \frac{12 nM}{{K}_{AF}} - 1 ]$$

yielding K_{AF}=0.30 nM where K_{F}=12 nM has been used. The value of K_{F} used to compute K_{AF} was independently obtained from activity data gathered for the pure homogeneous fucosylated antibody (Chung et al. 2012). The value of K_{AF} so computed is the first to appear in the public domain.

### Example 2: Computing K_{A}

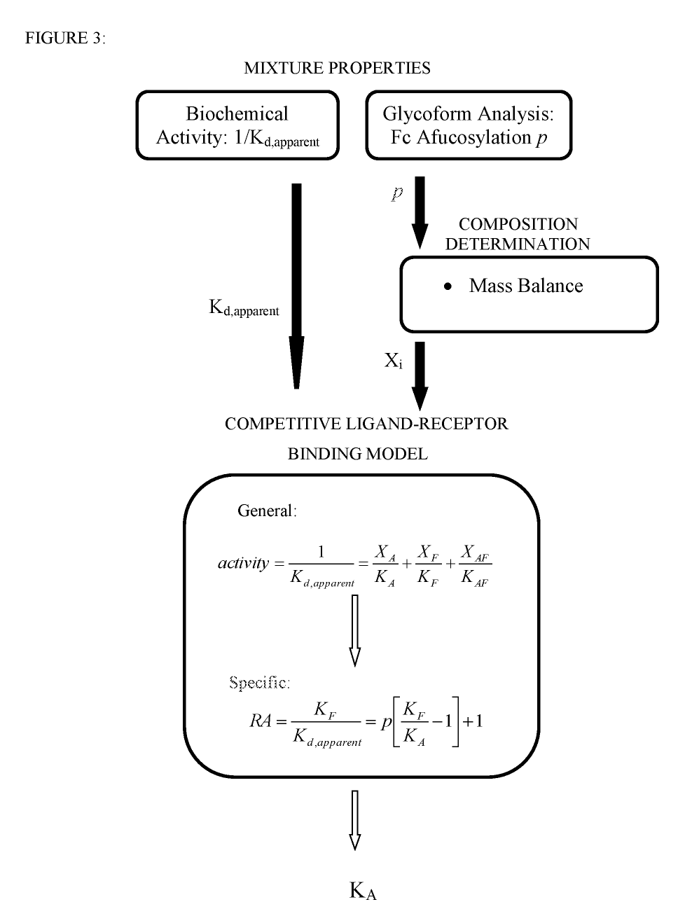

FIG. 3 shows the flowchart used to compute K_{A}, the dissociation equilibrium constant for the homogeneous afucosylated antibody, using activity obtained from mixtures. Chung and coworkers (2012) constructed mixtures comprised of defined proportions of the homogeneous fucosylated F and the homogeneous afucosylated A antibodies so as to arbitrarily set p in accordance with the relationship between p and mole fractions given by:

$$p = {X}_{A} + \frac{{X}_{AF}}{2} . \qquad ( 13 )$$

For example, a sample with p=0.5 was created by preparing an equimolar mixture of pure homogeneous afucosylated, X_{A}=1, and pure homogeneous fucosylated, X_{F}=1, antibodies. Since these artificial mixtures are characterized by the absence of the hemi-afucosylated form, or X_{AF}≈0, the binomial distribution cannot be used to compute mole fractions as in EXAMPLE 1. However, mole fractions may be obtained from afucosylated Fc glycan fractions p immediately from equation (13) by noting that p=X_{A} when X_{AF}≈0.

The appropriate mathematical model for analyzing the data of Chung and coworkers is given by:

$$\begin{matrix}Activity = \frac{1}{EC 50} = \frac{1}{{K}_{d , apparent}} = p [ \frac{1}{{K}_{A}} - \frac{1}{{K}_{F}} ] + \frac{1}{{K}_{F}} \\ ( {X}_{AF} \approx 0 ) & ( 14 ) \\ RA = \frac{EC {50}_{F}}{EC 50} = \frac{{K}_{F}}{{K}_{d , apparent}} = {X}_{A} [ \frac{{K}_{F}}{{K}_{A}} - 1 ] + 1 \\ ( {X}_{AF} \approx 0 ) . & ( 15 )\end{matrix}$$

Equation (15) is obtain from equation (14) by dividing equation (14) by 1/K_{F}. Equation (14) is obtained directly from equation (2) by noting that X_{AF}≈0 and using the mole fraction constraint

$${\sum }_{i = 1}^{m} {X}_{i} = 1. \qquad ( 16 )$$

Equations (14) predicts that activity or 1/K_{d,apparent} will scale linearly with p and that the slope and the y-intercept associated with this linear relationship are given by [1/K_{A}−1/K_{F}] and 1/K_{F} respectively. Similarly, the slope and the y-intercept associated with equation (15) are given by the terms [K_{F}/K_{A}−1] and 1 respectively. Therefore the existence of a linear relationship between mixture activity and p for artificial mixtures can be used to compute K_{A}.

Chung and coworkers (2012) reported on the existence of an empirical linear correlation between relative activity (RA), obtained from ELISA's measuring antibody Fc-FcγRIIIa F158 binding, and afucosylated Fc glycan fraction p for binary mixtures of homogeneous fucosylated and homogeneous afucosylated IgG1. Experimental RA data is obtained by dividing mixture activity, 1/K_{d,apparent}, by the activity of pure homogeneous fucosylated antibodies or 1/K_{F}. Experimental RA as defined is given by K_{d,apparent}/K_{F} in terms of the model parameters. Note that equations (14) and (15) theoretically predict the existence of a linear relationship between activity and afucosylation content. Therefore equation (15) is the appropriate mathematical model for analyzing the experimental relative activity data. Using the slope value of the empirical linear correlation reported by Chung and coworkers, 25, K_{A} can be computed using the relationship:

$$slope = 25 = [ \frac{{K}_{F}}{{K}_{A}} - 1 ] = [ \frac{12 nM}{{K}_{A}} - 1 ]$$

to yield K_{A}=0.46 nM where K_{F}=12 nM has been used. The value of K_{F} used to compute K_{A} was independently obtained from activity data gathered for the pure homogeneous fucosylated antibody (Chung et al. 2012).

### Example 3: Deconvolute Activity into Composition & Specific Activity

The competitive ligand-receptor binding mechanism provides the means to decompose mixture activity into component ligand contributions and to dissect component ligand contributions into composition and specific activity differences. For the afucosylated antibody system, steady state receptor binding activity is given by equation (2):

$$activity = \frac{1}{EC 50} = \frac{1}{{K}_{d , apparent}} = \frac{{X}_{A}}{{K}_{A}} + \frac{{X}_{F}}{{K}_{F}} + \frac{{X}_{AF}}{{K}_{AF}} . \qquad ( 2 )$$

Equation (2) reveals that the contribution to activity of each constituent antibody is the multiplicative product of the specific activity 1/K, and the mole fraction Xᵢ of the antibody. Therefore knowledge of the three dissociation equilibrium constants K_{A}, K_{F} and K_{AF} and the mole fractions X_{A}, X_{F} and X_{AF} is sufficient to completely and uniquely decomposed mixture activity. Due to the availability of pure homogeneous afucosylated and fucosylated antibodies, K_{A} and K_{F} are obtained using standard experimental methods. However the hemi-afucosylated antibody cannot be isolated in pure form so that K_{AF} can only be obtained using mathematical methods such as described in detail in EXAMPLE 1. Experimental limitations also preclude direct determination of the mole fractions of the different afucosylated antibody glycoforms necessitating use of the binomial distribution to compute compositions based on statistical considerations governing ligand assembly.

TABLE 1 shows the decomposition of mixture activity into the contributions to activity of the different afucosylated antibody glycoforms for different samples or mixtures. Column one shows the mixture activity for five different samples with different afucosylated Fc glycan fraction p (column two). Since activity is defined as 1/EC50 or 1/K_{d,apparent} it has units of reciprocal concentration. Columns 3-5 show how mixture activity is decomposed into the contributions of the different ligands. The last three columns show the mole fractions of the different glycoforms in the mixture computed using equation (8). The values of K_{A}, K_{F} and K_{AF} used to compute component glycoform activities are 0.46 nM, 12 nM and 0.30 nM respectively for the FcγRIIIa F158 allotype. K_{AF} and K_{A} were computed from experimental data as described in EXAMPLE 1 and EXAMPLE 2. K_{F} was obtained directly from experimental activity data for pure homogeneous fucosylated antibody. The data reveal that the both the hemi-afucosylated and the homogeneous afucosylated antibodies contribute significantly to activity.

TABLE 1
Component Antibody Activity for Different Ligand Mixtures

| Activity | | X_{A}/K_{A} | X_{F}/K_{F} | X_{AF}/K_{AF} | | | |
|---|---|---|---|---|---|---|---|
| [nM⁻¹] | p | [nm⁻¹] | [nM⁻¹] | [nm⁻¹] | X_{A} | X_{F} | X_{AF} |
| 0.08 | 0 | 0.0 | 0.08 | 0.0 | 0.0 | 1 | 0 |
| 0.41 | 0.05 | 0.0 | 0.075 | 0.33 | 0.0 | 0.90 | 0.10 |
| 0.74 | 0.1 | 0.0 | 0.07 | 0.67 | 0.0 | 0.80 | 0.20 |
| 2.36 | 0.9 | 1.76 | 0.0 | 0.6 | 0.81 | 0.01 | 0.18 |
| 2.17 | 1 | 2.17 | 0.0 | 0.0 | 1 | 0.0 | 0.0 |

### Example 4: Computing k_{on,AF} and k_{on,AF}

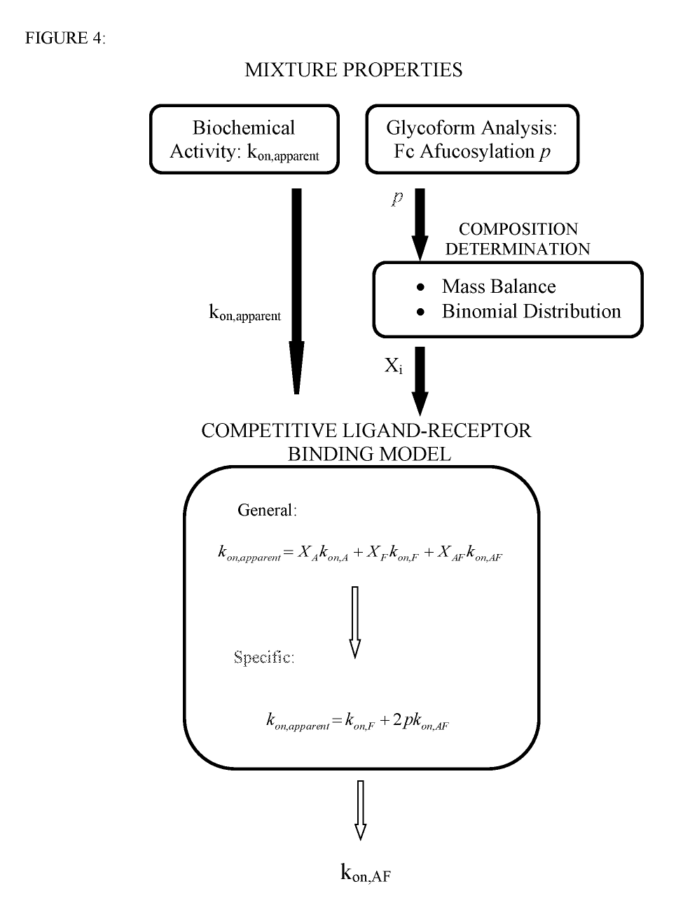

FIG. 4 shows the flowchart used to compute k_{on,AF} and k_{off,AF}, the forward and the reverse kinetic rate constants associated with the binding of the hemi-afucosylated antibody glycoform to receptor R. k_{on,apparent} and k_{off,apparent} obtained from mixtures are combined with mole fractions to compute component glycoform rate constants k_{on,i} and k_{on,i}. Rates constants for the ternary system comprising the three afucosylated antibody glycoforms are constrained by equation (4) as the direct consequence of the competitive binding mechanism:

kₒₙ=k_{on,apparent}=X_{A}k_{on,A}+X_{F}k_{on,F}+X_{AF}k_{on,AF} (4)

Using the binomial distribution with n=2, equation (8), to compute component antibody mole fractions yields:

kₒₙ=k_{on,apparent}=p²k_{on,A}(1−p)²k_{on,F}+2(p−p²)k_{on,AF} (17)

For low p mixtures, p≦0.1, equation (17) simplifies yielding:

k_{on,apparent}=k_{on,F}+2pk_{on,AF} (18).

Equation (18) predicts that k_{on,apparent} will vary linearly with afucosylated Fc glycan fraction p and that the slope and the y-intercept of this linear relationship are 2k_{on,AF} and k_{on,F} respectively. Therefore a plot of k_{on,apparent} versus p can be used to compute k_{on,AF}. k_{off,AF} may be straightforwardly computed from K_{AF} and k_{on,AF} using equation (5).

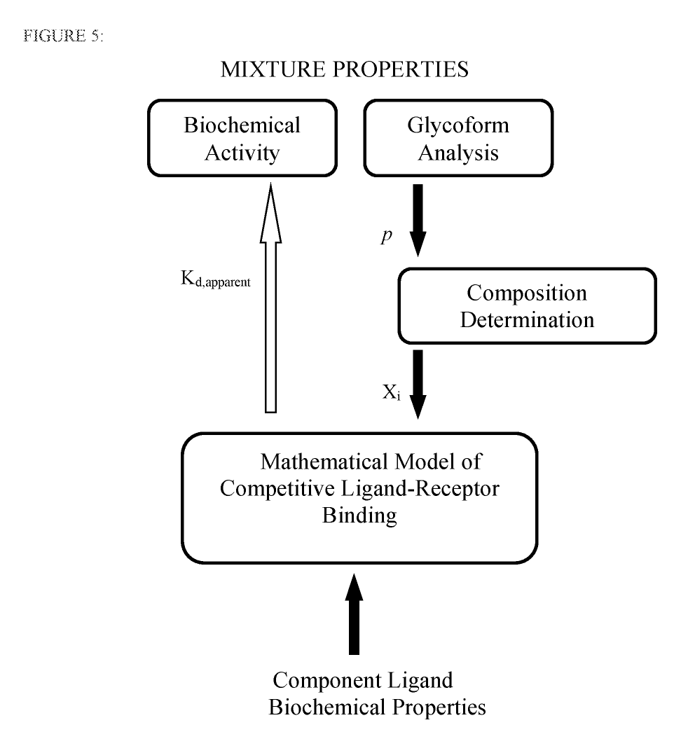

### DETAILED DESCRIPTION—SECOND EMBODIMENT—FIG. 5

FIG. 5 shows how mathematical models of competitive ligand-receptor binding are used to predict or compute mixture properties, such as K_{d,apparent}, using component ligand mole fractions, Xᵢ's, and properties. The main difference between this and the previously described embodiment is the flow of information described by the arrows in FIG. 5. The same mathematical models that are used to compute component properties can be used to compute mixture properties. Only the inputs, solid arrows, and the outputs, open arrows, into the mathematical equations have changed. Mole fractions and component ligand properties such as K₁ and k_{on,i}, are used to compute mixture properties such as the equilibrium constant K_{d,apparent} and the kinetic rate constant k_{on,apparent}. Component ligands compositions may be obtained from a number of sources including if possible direct experimental measurements or indirectly using statistical distributions and mass balances.

Also of interest is the variable f, the fraction receptors occupied. Experimentally, f represents the normalized dose response output obtained from classical ligand-receptor binding assays such as ELISAs and is defined as the ratio of the molar concentrations of total bound receptors [RL]ₜₒₜₐₗ to total receptors [R]ₜₒₜₐₗ or:

$$f = \frac{{[ RL ]}_{total}}{{[ R ]}_{total}} . \qquad ( 19 )$$

The appropriate equation for computing f from total ligand concentration [L]ₜₒₜₐₗ is:

$$f = \frac{{[ L ]}_{total}}{{[ L ]}_{total} + {K}_{d , apparent}} . \qquad ( 20 )$$

Equation (20) is obtained by combining the definition off the definition of K_{d,apparent}, the definitions of Kᵢ and the overall receptor molar balance:

[R]ₜₒₜₐₗ=[R]+[RL]ₜₒₜₐₗ.

For an m ligand system, the competitive ligand-receptor binding model allows f to be expressed as the sum of the component ligand contributions:

$$\begin{matrix}f = {\sum }_{i = 1}^{m} {f}_{i} \\ with & ( 21 ) \\ {f}_{i} = \frac{[ {L}_{i} ]}{[ {L}_{i} ] + {K}_{i} ( 1 + {{\sum }_{j = 1 , j \neq i}}^{m} \frac{[ {L}_{j} ]}{{K}_{j}} )} . & ( 22 )\end{matrix}$$

fᵢ denotes the fraction of overall available receptors occupied by ligand i. Equations (20) and (21) predict identical responses f. However equation (21) shows how f comprises the component ligand contributions. Since each fᵢ cannot in general be obtained experimentally, equations (21) and (22) are able to extract information from existing data that would otherwise remain unrevealed.

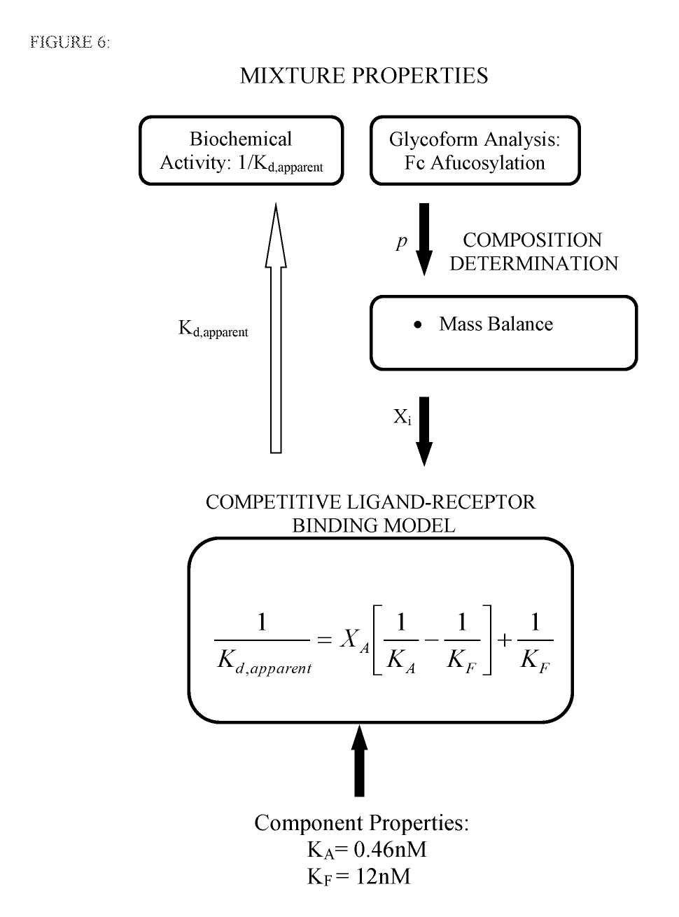

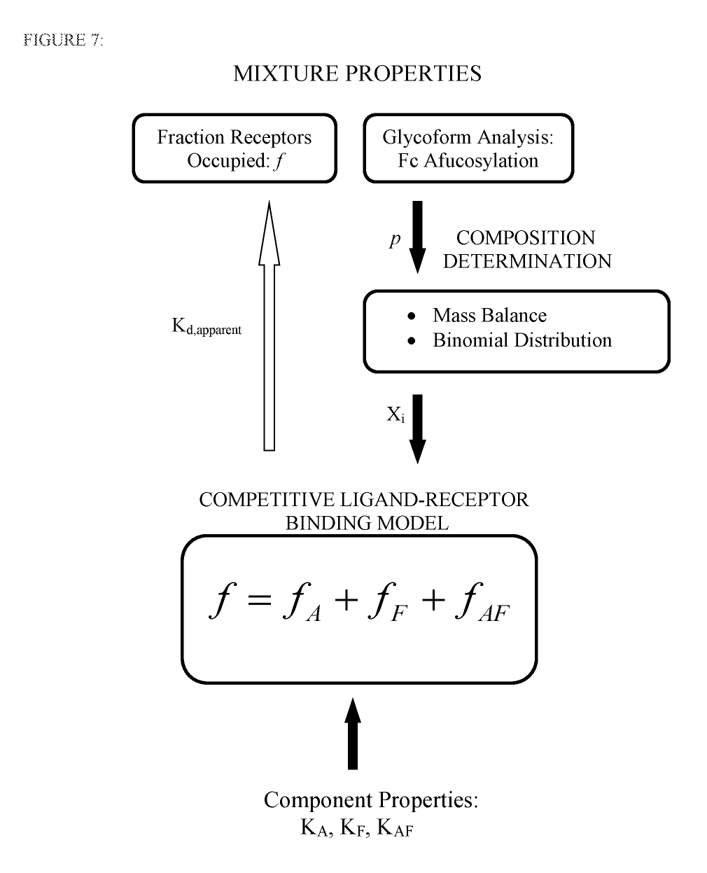

### OPERATIONS—SECOND EMBODIMENT—FIGS. 6 AND 7

Use of mathematical models of competitive ligand-receptor binding to compute mixture properties is outlined in the general following steps:
1) acquire component property information such as the dissociation equilibrium constants Kᵢ's,
2) acquire the mole fractions of the mixture components using: direct measurements, statistical distributions, mass balances or combinations of these elements,
3) obtain the specific form the general mathematical model of competitive ligand-receptor binding using experimental considerations,
4) use mathematical model to compute mixture property.

### Example 1: Mixtures of Homogeneous Antibodies

For binary mixture of homogeneous fucosylated and homogeneous afucosylated antibodies that compete for the common receptor FcγRIIIa (CD16a), equation (14) is the specific form of equation (2) that applies. Since X_{AF}≈0 for this system, p is given by X_{A}. FIG. 6 illustrates how equation (14) is used to compute mixture activity from component antibody equilibrium constants, K_{A} and K_{F}, with mole fraction X_{A}, or equivalently p, variable.

TABLE 2 shows computed and experimental values of 1/K_{d,apparent} for the binary homogenenous system with activity determined using ELISA for IgG1 Fc-FcγRIIIa F158 and IgG1 Fc-FcγRIIIa V158 receptor binding. The mixtures comprise define proportions of homogeneous fucosylated and afucosylated IgG1. For binary mixtures comprising the homogeneous fucosylated and afucosylated antibodies, X_{A} is identically p. X_{F} is computed from the mole fraction constraint for a binary mixture. K_{A}=0.46 nM and K_{F}=12 nM for the FcγRIIIa F158 allotype and K_{A}=0.167 nM and K_{F}=167 nM for the FcγRIIIa V158 allotype.

TABLE 2
1/K_{d,apparent} Computed for Binary Mixtures of A and F
(Fc [symbol] RIIIa F158 & V158)

| | | | 1/K_{d,apparent} F | 1/K_{d,apparent} F | 1/K_{d,apparent} V | 1/K_{d,apparent} V |
|---|---|---|---|---|---|---|
| p | X_{A} | X_{F} | (computed) | (observed) | (computed) | (observed) |
| 0 | 0 | 1 | 0.083 | 0.083 | 0.6 | 0.6 |
| 0.02 | 0.02 | 0.98 | 0.132 | 0.12 | 0.71 | 0.63 |
| 0.05 | 0.05 | 0.95 | 0.20 | 0.14 | 0.87 | 0.75 |
| 0.075 | 0.075 | 0.925 | 0.26 | 0.21 | 1.0 | 1.0 |
| 0.1 | 0.1 | 0.9 | 0.33 | 0.19 | 1.1 | 1.2 |
| 0.2 | 0.2 | 0.8 | 0.57 | 0.38 | 1.7 | 1.4 |
| 0.5 | 0.5 | 0.5 | 1.3 | 1.3 | 3.3 | 3 |
| 1 | 1 | 0 | 2.5 | 2.5 | 6.0 | 6 |

### Example 2: Fraction Receptors Occupied

FIG. 7 shows how the fraction receptors occupied f is computed for the ternary afucosylated antibody system. The appropriate model equations are given by equation (20) and equations (21) and (22) with m=3. Adopting the appropriate subscripts to denote the three afucosylated antibody glycoforms, A, F and AF yields:

$$\begin{matrix}f = \frac{[ Ab ]}{[ Ab ] + {K}_{d , apparent}} = {f}_{A} + {f}_{F} + {f}_{AF} , \\ with & ( 23 ) \\ {f}_{A} = \frac{[ A ]}{[ A ] + {K}_{A} ( 1 + \frac{[ F ]}{{K}_{F}} + \frac{[ AF ]}{{K}_{AF}} )} \\ {f}_{F} = \frac{[ F ]}{[ F ] + {K}_{F} ( 1 + \frac{[ A ]}{{K}_{A}} + \frac{[ AF ]}{{K}_{AF}} )} \\ {f}_{AF} = \frac{[ AF ]}{[ AF ] + {K}_{AF} ( 1 + \frac{[ A ]}{{K}_{A}} + \frac{[ F ]}{{K}_{F}} )} . & ( 24 )\end{matrix}$$

When supplied with component ligand equilibrium constants K_{A}, K_{F} and K_{AF} and compositions, equations (23) and (24) can be used to compute both f and the component ligand contributions fᵢ as of function of overall and individual ligand concentrations.

The concentrations appearing in equation (23) and equation (24) denote the concentrations of unbound antibody. The general excess of antibody or ligand over receptor allows the antibody concentrations appearing in the equations to be approximated by the antibody concentration added to the experimental samples. Unless otherwise noted, the numerical values used for antibody concentrations are assumed to be equal to the antibody concentrations added to the sample.

### DESCRIPTION—ADDITIONAL EMBODIMENT

Ligand-receptor binding is a specific class of protein-protein interactions involving one binding partner that has been termed a ligand and the other binding partner that has been termed a receptor. However equation (1) does not depend on any particular linguistic classification of proteins or entities and describes any system involving m proteins or entities that bind competitively to a common partner. Accordingly, equation (1) may be used to characterize any mixture of proteins or entities that competitively bind to a common entity such as the binding of mixtures of enzymes to a common protein. When the components of the mixture are multimers with each multimer comprised of k monomers with each monomer differentiated by the presence or absence of a defined molecular entity at a specific site on the monomer, then k+1 different multimers exist and the binomial distribution with n=k can be used to compute the mole fractions of the k+1 different forms.

For example, consider a dimeric enzyme comprised of two monomers with each monomer containing one site that may or may not contain a bound mannose-6-phosphate molecule. Statistical considerations give rise to three different glycoforms with mole fractions given by equation (8). The applicability of equation (8) follows immediately from the fact that an antibody molecule is a dimer. Simply replace the term “antibody” with “dimer.” In an assay where the different glycoforms competitively bind to a common protein, such as a receptor R, the applicability of equation (1) follows immediately.

### Mathematical Nomenclature

EC50 Experimental ligand concentration that induces a 50% maximum response
EC50_{F} Homo. fucosylated antibody concentration that induces a 50% max. response
f Fraction of total receptor bound to all ligands
f_{A} Fraction of total receptors bound to homogeneous afucosylated antibody
f_{AF} Fraction of total receptors bound to hemi-afucosylated antibody
f_{F} Fraction of total receptors bound to homogenous fucosylated antibody
fᵢ Fraction of total receptors bound to ligand i
f_{A}* Fraction of total bound receptors bound to homogeneous afucosylated antibody
f_{AF}* Fraction of total bound receptors bound to hemi-afucosylated antibody
f_{F}* Fraction of total bound receptors bound to homogeneous fucosylated antibody
fᵢ* Fraction of total bound receptors bound to ligand i
k_{off} dissociation reaction kinetic rate constant
k_{off,apparent} apparent dissociation reaction kinetic rate constant
k_{off,AF} dissociation reaction kinetic rate constant for hemi-afucosylated antibody
k_{off,F} dissociation reaction kinetic rate constant for homogeneous fucosylated antibody
k_{off,i} dissociation reaction kinetic rate constant for ligand i
kₒₙ binding reaction kinetic rate constant
k_{on,apparent} apparent binding reaction kinetic rate constant
k_{on,A} binding reaction kinetic rate constant for homogeneous afucosylated antibody
k_{on,AF} binding reaction kinetic rate constant for hemi-afucosylated antibody
k_{on,F} binding reaction kinetic rate constant for homogeneous fucosylated antibody
K_{d,apparent} apparent dissociation equilibrium constant
K_{A} dissociation equilibrium constant for homogeneous afucosylated antibody
K_{AF} dissociation equilibrium constant for hemi-afucosylated antibody
K_{F} dissociation equilibrium constant for homogeneous fucosylated antibody
Kᵢ dissociation equilibrium constant for ligand i
p fraction of Ig Fc heavy chains that are afucosylated
RA relative activity or activity relative to 1/K_{F}
rₒₙ rate of binding of all ligands to receptor
r_{on,i} rate of binding of ligand i to receptor
X_{A} mole fraction of unbound homogeneous afucosylated antibody
X_{AF} mole fraction of unbound hemi-afucosylated antibody
X_{F} mole fraction of unbound homogeneous fucosylated antibody
Xᵢ mole fraction of unbound ligand i
[A] molar concentration of unbound homogeneous afucosylated antibody
[AF] molar concentration of unbound hemi-afucosylated antibody
[F] molar concentration of unbound homogeneous fucosylated antibody
[Ab]ₜₒₜₐₗ molar concentration of total unbound antibody
[L]ₜₒₜₐₗ molar concentration of total unbound ligand
[Lᵢ] molar concentration of unbound ligand i
[R] molar concentration of unbound receptor
[R]ₜₒₜₐₗ molar concentration of total bound and unbound receptor
[RAb]ₜₒₜₐₗ molar concentration of total bound receptor
[RLᵢ] molar concentration of receptor bound to ligand i
[RL]ₜₒₜₐₗ molar concentration of total bound receptor

### CONCLUSIONS, RAMIFICATIONS & SCOPE

The reader will see that use of a mechanism based mathematical model of competitive ligand-receptor binding enhances the ability to characterize mixtures of glycoform ligands providing access to fundamental information on the activities and compositions of important constituent glycoproteins that cannot be obtained by existing methods. Specifically, the methods described provide the means to extract biochemical property information for important biologically activity constituents in mixtures that currently cannot be isolated in pure form. Since mixtures of ligands are routinely employed therapeutically, the methods developed directly address an unmet need associated with biologics characterization. The utility of the methods developed was demonstrated with mixtures of afucosylated antibodies comprised of the three antibody glycoforms that are differentiated by afucosylated Fc glycan content. In addition to serving as a model system for methods development, the antibody afucosylation system is directly relevant to many marketed therapeutics that rely of Ig Fc mediated effector function. The methods developed provide the means to deduce the equilibrium constant K_{AF} which has evaded researchers hitherto.

The methods developed have the potential to raise the standard of quality for manufactured biologics worldwide by providing fundamental knowledge on the compositions and the specific activities of the important biochemically active glycoforms in therapeutic mixtures of ligands. Emphasis can now be placed on determining the distribution of glycoproteins or glycoforms in a mixture rather than their bound carbohydrates structures. Since the glycoform is the physical entity that mediates in vivo efficacy, the importance of this information cannot be overstated. Current methods cannot distinguish between glycan and glycoform compositions so that different manufacturers of the same glycoprotein must argue for glycoprotein similarity without fundamental information on the molar concentrations of the important constituent glycoforms. The methods developed in this invention provide manufacturers of biologics with the means to overcome this critical barrier to proper product characterization thus promoting higher standards of product quality.

Although the above descriptions contain many specifications, these should not be construed as limitations on the scope, but rather as examples of several embodiments thereof. Accordingly the scope of this invention should not be determined by the embodiment(s) illustrated, but by the appended claims and their legal equivalents.

We claim:

1. A method for characterizing a constituent ligand in a mixture of ligands in terms of a property of said ligand comprising:
a. measuring said property of said mixture,
b. using a mathematical model of competitive ligand-receptor binding with said model using said property of said mixture to compute said property of said ligand.

2. The method in claim 1 wherein said ligand is an antibody molecule.

3. The method in claim 1 wherein said property is an equilibrium constant and said model is
$$\frac{1}{{K}_{d , apparent}} = \frac{{X}_{1}}{{K}_{1}} + \frac{{X}_{2}}{{K}_{2}} + … \frac{{X}_{m - 1}}{{K}_{m - 1}} + \frac{{X}_{m}}{{K}_{m}}$$
or a mathematical equivalent.

4. The method in claim 1 wherein said property is a kinetic rate constant and said model is
k_{on,apparent}=X₁k_{on,1}+X₂k_{on,2}+ . . . +Xₘ₋₁k_{on,m-1}+Xₘk_{on,m} or
k_{off,apparent}=f₁*k_{off,1}+f₂*k_{off,2}+fₘ₋₁*k_{off,m-1}+fₘ*k_{off,m},
or a mathematical equivalent.

5. The method in claim 1 wherein said mixture comprises antibody molecules and said ligand is an antibody molecule and said model is
$$\frac{1}{{K}_{d , apparent}} = \frac{{X}_{1}}{{K}_{1}} + \frac{{X}_{2}}{{K}_{2}} + … \frac{{X}_{m - 1}}{{K}_{m - 1}} + \frac{{X}_{m}}{{K}_{m}}$$
or a mathematical equivalent further including use of the binomial distribution to compute the composition of said mixture.

6. A method for characterizing a constituent ligand in a mixture of ligands in terms of a property of said ligand in said mixture comprising:
a. measuring said property of said mixture,
b. using the binomial distribution to compute the composition of said mixture,
c. using a mathematical model of competitive ligand-receptor binding with said model using said property of said mixture and said composition to compute said property of said ligand.

7. The method in claim 6 wherein said ligand is an antibody molecule and said property is an equilibrium constant and said model is
$$\frac{1}{{K}_{d , apparent}} = \frac{{X}_{1}}{{K}_{1}} + \frac{{X}_{2}}{{K}_{2}} + … \frac{{X}_{m - 1}}{{K}_{m - 1}} + \frac{{X}_{m}}{{K}_{m}}$$
or a mathematical equivalent.

8. The method in claim 6 wherein said ligand comprises a plurality of subunits with said subunits having identical amino acid sequences.

9. The method in claim 6 wherein said ligand in an antibody molecule and said property is a kinetic rate constant and said model is
k_{on,apparent}=X₁k_{on,1}+X₂k_{on,2}+ . . . +Xₘ₋₁k_{on,m-1}+Xₘk_{on,m} or
k_{off}=k_{off,apparent}=f₁*k_{off,1}+f₂*k_{off,2}+fₘ₋₁*k_{off,m-1}+fₘ*k_{off,m}
or a mathematical equivalent.

10. The method in claim 6 wherein said mixture comprises homogeneous fucosylated antibody, hemi-afucosylated antibody and homogeneous afucosylated antibody and said ligand is an antibody molecule and said model is
$$\frac{1}{{K}_{d , apparent}} = \frac{{X}_{A}}{{K}_{A}} + \frac{{X}_{F}}{{K}_{F}} + \frac{{X}_{AF}}{{K}_{AF}}$$
or
kₒₙ=k_{on,apparent}=X_{A}k_{on,A}+X_{F}k_{on,F}+X_{AF}k_{on,AF} or
k_{off}=k_{off,apparent}=f_{A}k_{off,A}+f_{F}k_{off,F}+f_{AF}k_{off,AF}
or a mathematical equivalent.

11. A method for computing a property of a mixture of ligands comprising:
a. determining said property of constituent ligands in said mixture,
b. determining the composition of said mixture,
c. using a mathematical model of competitive ligand-receptor binding with said model using said property of said ligands and said composition to compute said property of said mixture.

12. The method in claim 11 wherein said property of said mixture is an equilibrium constant and said model is
$$\frac{1}{{K}_{d , apparent}} = \frac{{X}_{1}}{{K}_{1}} + \frac{{X}_{2}}{{K}_{2}} + … \frac{{X}_{m - 1}}{{K}_{m - 1}} + \frac{{X}_{m}}{{K}_{m}}$$
or a mathematical equivalent.

13. The method in claim 11 wherein said property of said mixture is a kinetic rate constant and said model is
k_{on,apparent}=X₁k_{on,1}+X₂k_{on,2}+ . . . +Xₘ₋₁k_{on,m-1}Xₘk_{on,m} or
k_{off}=k_{off,apparent}=f₁*k_{off,1}+f₂*k_{off,2}+fₘ₋₁*k_{off,m-1}+fₘ*k_{off,m}
or a mathematical equivalent.

14. The method in claim 11 wherein said mixture comprises antibody molecules and said model is
$$\frac{1}{{K}_{d , apparent}} = \frac{{X}_{1}}{{K}_{1}} + \frac{{X}_{2}}{{K}_{2}} + … \frac{{X}_{m - 1 }}{{K}_{m - 1}} + \frac{{X}_{m}}{{K}_{m}}$$
or
k_{on,apparent}=X₁k_{on,1}+X₂k_{on,2}+ . . . +Xₘ₋₁k_{on,m-1}+Xₘk_{on,m} or
k_{off}=k_{off,apparent}=f₁*k_{off,1}+f₂*k_{off,2}+fₘ₋₁*k_{off,m-1}+fₘ*k_{off,m}
or a mathematical equivalent.

15. The method in claim 11 wherein said mixture comprises homogeneous fucosylated antibody, hemi-afucosylated antibody and homogeneous afucosylated antibody and said model is
$$\frac{1}{{K}_{d , apparent}} = \frac{{X}_{A}}{{K}_{A}} + \frac{{X}_{F}}{{K}_{F}} + \frac{{X}_{AF}}{{K}_{AF}}$$
or
kₒₙ=k_{on,apparent}=X_{A}k_{on,A}+X_{F}k_{on,F}+X_{AF}k_{on,AF} or
k_{off}=k_{off,apparent}=f_{A}k_{off,A}+f_{F}k_{off,F}+f_{AF}k_{off,AF}
or a mathematical equivalent.

16. The method in claim 11 wherein said property of said mixture is the fraction receptors occupied f wherein said model comprises
$$f = {\sum }_{i = 1}^{m} {f}_{i} and {f}_{i} = \frac{[ {L}_{i} ]}{[ {L}_{i} ] + {K}_{i} ( 1 + {\sum }_{j = 1 , j \neq i}^{m} \frac{[ {L}_{j} ]}{{K}_{j}} )}$$
or a mathematical equivalent.

17. The method in claim 11 wherein said mixture comprises antibodies and said property of said mixture is the fraction receptors occupied f wherein said model comprises
$$f = {\sum }_{i = 1}^{m} {f}_{i} and {f}_{i} = \frac{[ {L}_{i} ]}{[ {L}_{i} ] + {K}_{i} ( 1 + {\sum }_{j = 1 , j \neq i}^{m} \frac{[ {L}_{j} ]}{{K}_{j}} )}$$
or a mathematical equivalent further including use of the binomial distribution to compute said composition of said mixture.
